(19) United States
(12) Patent Application Publication
(10) Pub. No.: US 2005/0276840 A1
(43) Pub. Date: Dec. 15, 2005

(54) Method and system for weight management

(75) Inventor: Scott Mann, Stow, OH (US)

Correspondence Address: Heather M. Barnes;Brouse McDowell, LPA, Ste. 500, 388 S. Main Street, Akron, OH, 44311-4407, US

(73) Assignee: Scott Mann Enterprises

(21) Appl. No.: 11/152,933

(22) Filed: Jun. 15, 2005

Related U.S. Application Data
(60) Provisional application No. 60/579,968, filed on Jun. 15, 2004.

(51) Int. Cl. A61K 47/00

(52) U.S. Cl. 424/439

(57) ABSTRACT

The present invention is a method for losing weight and optimizing body composition comprising the steps of eating five meals a day with 1 serving of vegetables or fruit, complex carbohydrate, and lean protein for approximately 300 calories per meal at ranges of 25-35% protein, 50-55% carbohydrate, 15-20% fat with six to seven grams of fiber for a total of 1,500 calories per day for women. The method also comprises a method for losing weight and optimizing body composition for men comprising the steps of eating five meals a day every 2 1/2 to 3½ hours consisting of 1½ servings of vegetables or fruit, complex carbohydrate, and lean protein for 450 calories per meal at 25-35% protein, 50-55% carbohydrate, and 15-20% fat with nine to ten grams of fiber for a total of 2,250 calories per day.

This application claims priority from a provisional patent application having Ser. No. 60/579,968, which was filed on Jun. 15, 2004.

### I. BACKGROUND OF THE INVENTION

A. Field of Invention

The present invention is directed to a method and system for weight management and body composition management. More specifically, the present invention is directed to weight loss and weight maintenance based upon a plurality of principles to optimize a person's metabolism, so as to favorably alter a person's body composition.

B. Description of the Related Art

Weight loss and weight management is a multi-billion dollar a year industry. With the majority of Americans being overweight, most people are looking for the “quick fix” in order to lose weight very quickly without starvation. Most systems known in the art are based on a reduced calorie intake. Other diet fads in the industry include a high fat, high protein, low carbohydrate ratio. There is a need in the art for a weight management program that enables a person to eat numerous meals throughout the day, to increase total caloric intake per day in order to increase muscle mass and lose fat in order to improve overall health.

It is generally accepted in the art that a woman's optimal percentage of fat relative to total body composition ranges from 20% to 23%. The range may increase up to 25% for women who are post menopausal. The ideal range for men is between 12% and 15%.

### II. SUMMARY OF THE INVENTION

The present invention is a method for losing weight comprising the steps of eating at least five meals a day every 2½ to 3½ hours with 1 serving of vegetables or fruit, 1 serving of complex carbohydrate, and 1 serving of lean protein for approximately 300 calories per meal at ranges of 25-35% protein, 50-55% carbohydrate, 15-20% fat with six to seven grams of fiber for a total of 1,500 calories per day for women. The method also comprises a method for losing weight for men comprising the steps of eating five meals a day every 2½ to 3½ hours consisting of 1½ servings of vegetables or fruit, 1½ servings of complex carbohydrate, and 1½ servings of lean protein for 450 calories per meal at 25-35% protein, 50-55% carbohydrate, and 15-20% fat with nine to ten grams of fiber for a total of 2,250 calories per day.

It is another object of the present invention to provide a weight management program that is an 80/20 ratio, meaning that the weight loss system can be followed 80% of the time in order to maintain a person's body weight.

It is another object of the present invention to provide a weight management system which resets a person's fat set point, increases muscle mass, and raises a person's metabolism.

It is yet another object of the present invention to provide a weight management system that is healthy and safe.

It is yet another object of the present invention to provide a weight management system that is realistic and easy to follow.

Further, another object of the present invention is to provide a weight management system that utilizes common, every day foods found in most grocery stores.

It is yet another object of the present invention to provide a weight management system that assists the user to maintain healthy lifestyle changes.

It is yet another object of the present invention to provide recipes for foods that include the proper ratio and caloric intake per serving.

It is yet another object of the present invention to provide recipes for muffins, meal replacement bars, fruit yogurts, turkey chili, chicken potato soup, turkey chili with pasta, whole wheat pizza, whole wheat pita pizza, and chicken potato and pasta soup.

It is yet another object of the present invention to provide recipes for meals having the above described ratios, wherein such meals are sold through food programs, such as at schools and restaurants.

It is yet another object of the present invention to provide the meals such that 300 calories is equivalent to a small size serving, 450 calories is equivalent for a medium size serving, and 600 calories is equivalent for a large size serving.

Another object of the present invention is to provide a method for managing weight, comprising the steps of: eating a meal at least five times a day with one serving of a vegetables or a fruit, a complex carbohydrate, and a lean protein for a predetermined number of calories per meal at ranges of 25-35% protein, 50-55% carbohydrate, 15-20% fat with a predetermined range of grams of fiber; losing weight; and optimizing body composition.

Another object of the present invention is to provide a method for managing weight, wherein the predetermined number of calories is about 300 calories and the predetermined range of grams of fiber is about six to seven grams.

Another object of the present invention is to provide a method for managing weight, wherein the predetermined number of calories is about 450 calories and the predetermined range of grams of fiber is about nine to ten grams of fiber.

Another object of the present invention is to provide a method for managing weight, wherein the step of eating a meal at least five times a day further comprises the step of eating one food that includes 25-35% protein, 50-55% carbohydrate, 15-20% fat.

Another object of the present invention is to provide a method for managing weight, wherein the one food is selected from the group a muffin/bar, fruit yogurts, chicken potato soup, chicken potato & pasta soup, turkey chili, turkey chili & pasta, chicken whole wheat burrito, whole wheat pizza, or whole wheat pita pizza.

Another object of the present invention is to provide a method for managing weight, wherein the muffin/bar is a pumpkin muffin/bar comprising about 50 grams of egg whites, about 16.5 grams of whey protein, about 16.5 grams of rolled oats, about 3 grams of cornstarch, about 3 grams ground flax seed, about 6 grams of almond flour, about 2.5 grams of oat bran, about 115 grams of pumpkin, about 7.5 grams of fructose, about 1.5 grams of honey, about ⅔ teaspoon of pumpkin pie spice, about ⅔ teaspoon of cinnamon, about ¼ teaspoon of baking powder, about ⅛ teaspoon salt, about ½ teaspoon of calcium powder, and about 1/16 teaspoon of baking soda, so as to result in about one serving.

Another object of the present invention is to provide a method for managing weight, wherein the muffin/bar is an apple cinnamon muffin/bar, comprising about 50 grams of egg whites, about 17 grams of whey protein, about 16 grams of rolled oats, about 3 grams of cornstarch, about 3 grams of ground flax seed, about 6 grams of almond flour, about 3 grams of oat bran, about 30 grams of apple sauce, about 56 grams of apples, about 8 grams of fructose, about ⅔ teaspoon of apple pie spice, about ⅔ teaspoon of cinnamon, about ¼ teaspoon of baking powder, about ⅛ teaspoon of salt, about ½ teaspoon of calcium powder, and about 1/16 teaspoon of baking soda, so as to result in about one serving.

Another object of the present invention is to provide a method for managing weight, wherein the muffin/bar is a blueberry muffin/bar, comprising about 50 grams of egg whites, about 16.5 grams of whey protein, about 17 grams of rolled oats, about 3.5 grams of cornstarch, about 3 grams of ground flax seed, about 6 grams of almond flour, about 5 grams of oat bran, about 20 grams of apple sauce, about 42 grams of blueberries, about 8.5 grams of fructose, about ¼ teaspoon of baking powder, about ⅛ teaspoon of salt, about ½ teaspoon calcium powder, and about 0.25 grams of xanthan gum, so as to result in about one serving.

Another object of the present invention is to provide a method for managing weight, wherein the muffin/bar is a banana-nutmeg muffin/bar comprising about 50 grams of egg whites, about 16.5 grams of whey protein, about 16.5 grams of rolled oats, about 2.5 grams of cornstarch, about 3 grams of ground flax seed, about 6 grams of almond flour, about 3 grams of oat bran, about 58 grams of bananas, about 7 grams of fructose, about ½ teaspoon of vanilla, about ¼ teaspoon of allspice, about ¼ teaspoon of nutmeg, about ¼ teaspoon of baking powder, about ⅛ teaspoon of salt, about ½ teaspoon of calcium powder, and about 1/16 teaspoon of baking soda, so as to result in about one serving.

Another object of the present invention is to provide a method for managing weight, wherein the muffin/bar is a cherry-vanilla muffin/bar comprising about 50 grams of egg whites, about 16.5 grams of whey protein, about 16 grams of rolled oats, about 3 grams of cornstarch, about 3 grams of ground flax seed, about 6 grams of almond flour, about 5 grams of oat bran, about 15 grams of yogurt, about 58 grams of cherries, about 6 grams of fructose, about one teaspoon vanilla, about ¼ teaspoon baking powder, about ⅛ teaspoon salt, about ½ teaspoon of calcium powder, and about 1/16 teaspoon baking soda, so as to result in about one serving.

Another object of the present invention is to provide a method for managing weight, wherein the chicken potato soup comprises about 56 grams of chicken breast, cooked by dry heat without skin, about 142 grams of a boiled potato, about 85 grams of a raw carrot, about 56 grams of diced raw celery, about 40 grams of a chopped raw onion, about 2.5 grams of extra virgin olive oil, about 2 chicken bouillon cubes, and about 2¼ cups water, wherein the chicken breast, potato, carrot, celery, onion, olive oil, bouillon, and water are heated to result in about one single serving.

Another object of the present invention is to provide a method for managing weight, wherein the chicken potato and pasta soup comprises about 56 grams of chicken breast, about 71 grams of a boiled potato, about 42 grams of pasta-durum semolina, about 85 grams of a carrot, about 56 grams of celery, about 40 grams of an onion, about 2½ grams of olive oil, about 2 chicken bouillon cubes, about 2¼ cups of water, wherein the chicken breast, potato, pasta, carrot, celery, onion, olive oil, bouillon cubes, and water are heated to result in about one single serving.

Another object of the present invention is to provide a method for managing weight, wherein the turkey chili comprises about 2 ounces lean ground turkey, about 5.9 ounces of kidney beans, about 2.81 ounces of tomato sauce, about 3.62 ounces of diced tomatoes, about 2 ounces of an onion, and about .24 ounces of chili seasoning to result in about one single serving.

Another object of the present invention is to provide a method for managing weight, wherein turkey chili and pasta comprises about 2.5 ounces lean ground turkey, about 1.5 ounces of pasta, about 2.38 ounces of kidney beans, about 3.75 ounces of tomato sauce, about 3.63 ounces of diced tomatoes, about .75 ounces of green peppers, about 0.75 ounces of an onion, and about 0.24 ounces of chili seasoning to result in about one single serving.

Another object of the present invention is to provide a method for managing weight, wherein the chicken whole wheat burrito comprises about 43 grams of chicken breast roasted without skin, about 65 grams of a whole wheat tortilla, about 35 grams of cooked long grain rice, about 28 grams of cooked black beans, and about 99 grams of raw chopped tomatoes so as to result in about one single serving.

Another object of the present invention is to provide a method for managing weight, wherein the whole wheat burrito substitutes about 1.75 ounces of lean sirloin beef or about 2 ounces of Mahi Mahi fish for the chicken breast.

Another object of the present invention is to provide a method for managing weight, wherein the whole wheat pita pizza comprises about 1.5 ounces of cubed chicken breast, about 2.25 ounces of pita bread, about 2 ounces of low fat pasta sauce, about 2.25 ounces of sliced mushrooms, and about .5 ounce of reduced fat cheese so as to result in about one single serving.

Another object of the present invention is to provide a method for managing weight, wherein the chicken breast can be substituted with about 2 ounces of lean turkey sausage, about 2 ounces of lean ground turkey, or about 3 ounces of Canadian bacon.

Another object of the present invention is to provide a method for managing weight, wherein the mushrooms can be substituted with about 1.4 ounces of onion, about 1.8 ounces of sweet peppers, or about 2.4 ounces of tomato.

Another object of the present invention is to provide a method for managing weight, wherein the deluxe whole wheat pizza comprises about 11 grams of mushrooms, about 17 grams of sweet pepper, about 17 grams of onion, about 51 grams of low-fat pasta/pizza sauce, about 56 grams of whole wheat pizza crust, about 56 grams of lean turkey sausage, and about 0.80 grams of fat-free mozzarella cheese so as to result in about one single serving.

Another object of the present invention is to provide a method for managing weight, wherein the lean turkey sausage can be substituted with about 2 ounces of ground turkey or 1.5 ounces of chicken breast.

Another object of the present invention is to provide a method for managing weight, wherein the fruit yogurt comprises about 19 grams of whey protein, about 220 grams of low-fat vanilla yogurt, about 99 grams of strawberries, and about 4 grams of fructose so as to result in about one single serving.

Another object of the present invention is to provide a method for managing weight, wherein the strawberries can be substituted with about 50 grams of apples, about 54 grams of blueberries, about 45 grams of cherries, about 70 grams of peaches, about 54 grams of pineapple, and about 61 grams of raspberries.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, a preferred embodiment of which will be described in detail in this specification and illustrated in the accompanying drawings which form a part hereof and wherein:

FIG. 1 is a chart showing preferred vegetables to be used with the present invention.

FIG. 1A is a chart showing preferred vegetables to be used with the present invention.

FIG. 2 is a chart showing preferred fruits to be used with the present invention.

FIG. 3 is a chart of preferred complex carbohydrates to be used with the present invention.

FIG. 3A is a chart of preferred complex carbohydrates to be used with the present invention.

FIG. 4 is a chart of preferred lean proteins to be used with the present invention.

FIG. 4A is a chart of preferred lean proteins to be used with the present invention.

FIG. 5 is a chart showing preferred condiments and dietary fats to be used in conjunction with the present invention.

FIG. 6 is a chart showing preferred seasoning to be used in conjunction with the present invention.

### DESCRIPTION OF THE PREFERRED EMBODIMENT

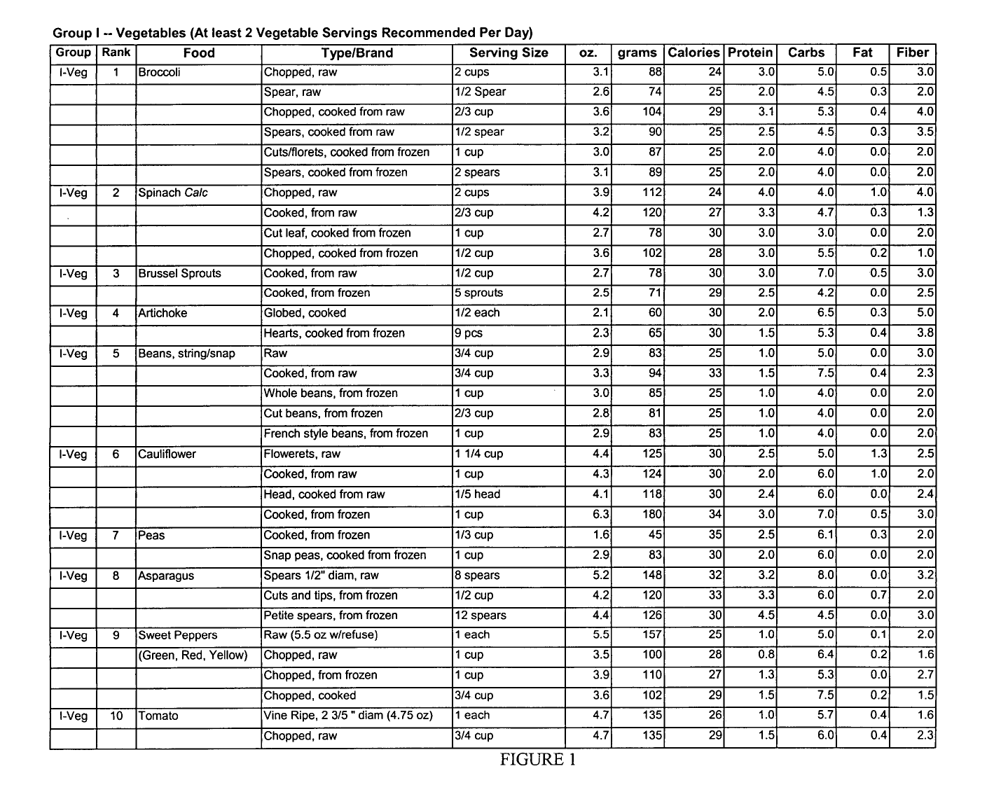

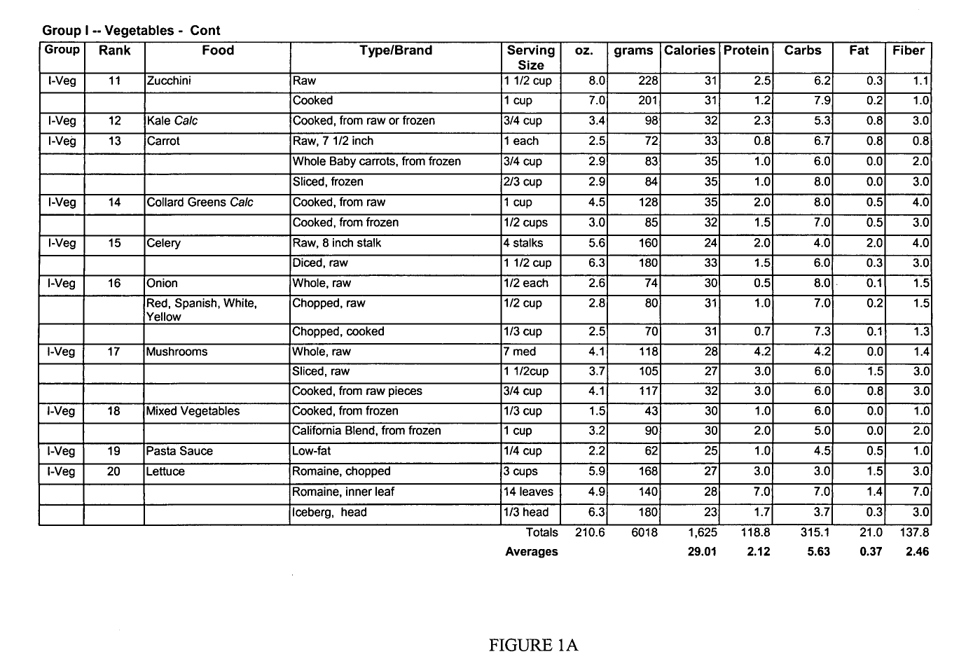

Referring now to the drawings wherein the showings are for purposes of illustrating a preferred embodiment of the invention only and not for purposes of limiting the same, FIGS. 1-6 aid in illustrating the present invention. The present invention is a weight management system based upon a nutritional exchange system that gives individuals the freedom and flexibility to eat a balanced variety of foods, while staying within the nutritional parameters needed to achieve optimal health, fitness and body composition.

Each food has a specific serving size and is categorized by Vegetables (Group I), Fruits (Group II), Complex Carbohydrates (Group III), Lean Proteins (Group IV), Condiments (Group V), and Seasonings (Group VI).

The nutritional guidelines of the inventive method and system are based upon the nutrition principles: 1) eat the right number of calories; 2) eat frequently throughout the day; 3) eat the right combination of nutrients; and 4) eat the right quality of food. The nutritional format of the present invention is based upon a single meal unit that contains approximately 300 calories (+ or − 10 calories) at 25-35% Protein (18.75-26.25 grams), 50-55% Carbohydrate (37.5-41.25 grams), and 15-20% fat (5.0-6.6 grams) with 6-7 grams of fiber. These nutritional objectives are met by consuming a “complete” meal that contains:

1 serving of Vegetable (Group I) or Fruit (Group II),
Veg serving avg.=29.01 cal, 2.12 g pro, 5.63 g carb, 0.37 g fat, 2.46 g fiber.
Fruit serving avg.=49.76 cal, 0.74 g pro, 12.17 g carb, 0.38 g fat, 2.04 g fiber.

1 serving of Complex Carbohydrate (Group III), and
Carb serving avg.=156.15 cal, 6.60 g pro, 31.12 g carb, 1.28 g fat, 4.31 g fiber.

1 serving of Lean Protein (Group IV).

Protein serving avg.=90 cal, 16.12 g pro, 0.26 g carb, 2.20 g fat, 0.37 g sat fat.

One serving of condiments/dietary fats (Group V)
Condiment/Fat avg.=32.78 cal, 0.77 g pro, 2.86 g carb, 1.96 g fat.

One serving of condiments/dietary fats in Group V can be added to complete meals to supplement low fat protein choices.

Women can use this base meal formula to meet their minimal daily caloric requirements by consuming 5 “complete” meals per day, each meal every 2½ to 3½ hours. Five “complete” meals per day with 1 serving each of Vegetables (Group I) or Fruit (Group II), with Complex Carbohydrates (Group III), and Lean Proteins (Group IV) averages out to approximately 1,500 calories per day at 25-35% protein, 50-55% carbohydrate, and 15-20% fat with 30-35 grams of fiber.

Women can adjust or increase their caloric intake first by adding a 6^{th} complete meal to their nutrition plan. This will raise their caloric intake to 1,800 calories per day. From there, calories for women can be further increased as needed by consuming 1½ servings each of Vegetables (Group I) or Fruit (Group II), Complex Carbohydrates (Group III), and Lean Proteins (Group IV) for one of the 6 daily meals. This will raise the caloric intake to 1,950 calories per day.

Increasing the serving size to 1½ servings per meal can be continued as follows:
2 of 6 meals at 1½ servings each=2,100 calories per day.
3 of 6 meals at 1½ servings each=2,250 calories per day.
4 of 6 meals at 1½ servings each=2,400 calories per day.
5 of 6 meals at 1½ servings each=2,550 calories per day.
All 6 meals at 1½ servings each=2,700 calories per day.

Men can use the base meal formula to meet their minimal daily caloric requirements by consuming 5 meals with 1½ servings of each of Vegetables (Group I) or Fruit (Group II), with Complex Carbohydrates (Group III), and Lean Proteins (Group IV) with each meal. “Complete” meals comprised of 1½ servings each contain approximately 450 calories at 25-35% protein, 50-55% carbohydrate, and 15-20% fat with 9-10 grams of fiber. Eating 5 “complete” meals every 2½ to 3½ hours averages out to approximately 2,250 calories per day at 25-35% protein, 50-55% carbohydrate, and 15-20% fat with 45-50 grams of fiber.

Men can adjust or increase their caloric intake first by adding a 6^{th} complete meal with 1½ servings each to their nutritional plan. This will raise their caloric intake to 2,700 calories per day. From there, calories for men can be adjusted or increased by consuming 2 servings of each of Vegetables (Group I) or Fruit (Group II), Complex Carbohydrates (Group III), and Lean Proteins (Group IV) for one of the 6 daily meals. This will raise the caloric intake to 2,850 calories per day.

Increasing the serving size to 2 servings per meal can be continued as follows:

2 of 6 meals at 2 servings each=3,000 calories per day.

3 of 6 meals at 2 servings each=3,150 calories per day.

4 of 6 meals at 2 servings each=3,300 calories per day.

5 of 6 meals at 2 servings each=3,450 calories per day.

All 6 meals at 2 servings each=3,600 calories per day.

With reference to FIGS. 1-6, foods are ranked in order of preference in order to maximize the benefits of the present invention.

With reference to FIGS. 1 and 1A, vegetables may be chosen from the group consisting of broccoli, spinach, brussel sprouts, artichokes, beans (string/snap), cauliflower, peas, asparagus, sweet peppers, tomatoes, zucchinis, kale, carrots, collard greens, celery, onions, mushrooms, mixed vegetables, pasta sauce, and lettuce.

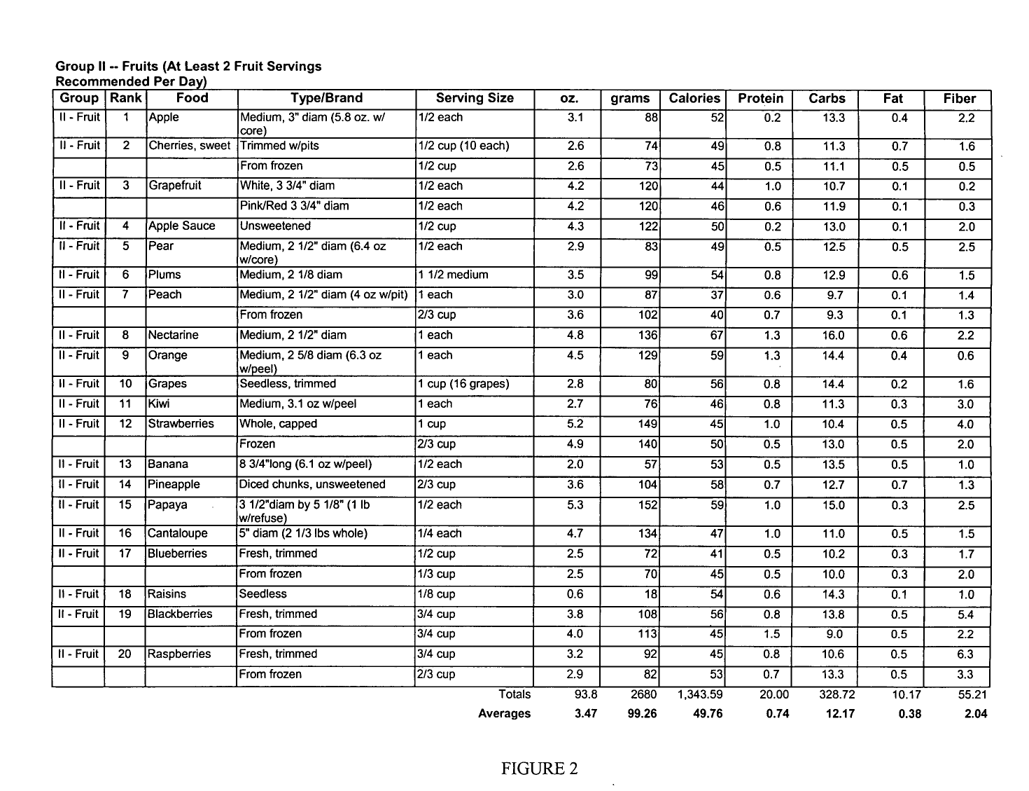

With reference to FIG. 2, fruits may be chosen from the group consisting of apples, sweet cherries, grapefruits, apple sauce, pears, plums, peaches, nectarines, oranges, grapes, kiwis, strawberries, bananas, pineapples, papayas, cantaloupes, blueberries, raisins, blackberries, and raspberries.

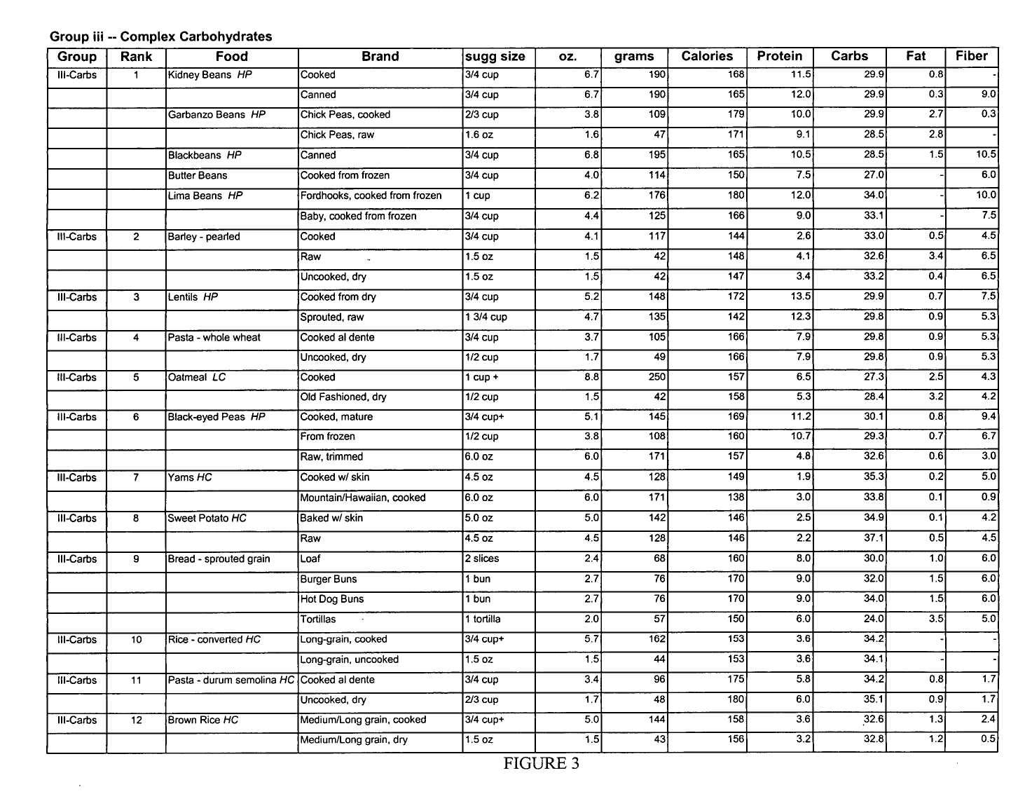

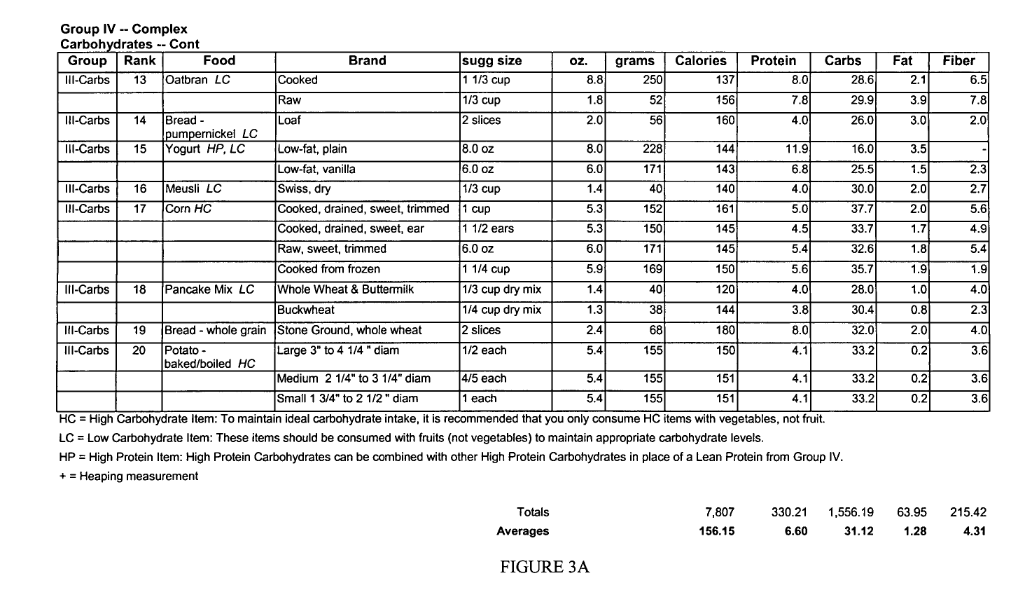

With reference to FIG. 3, complex carbohydrates may be chosen from the group consisting of kidney beans, garbanzo beans, black beans, butter beans, lima beans, pearled barley, lentils, whole wheat pasta, oatmeal, back-eyed peas, yams, sweet potatoes, spouted grain bread, converted rice, durum semolina pasta, brown rice, oat bran, pumpernickel bread, yogurt muesli, corn, pancake mix, whole grain bread, and baked or boiled potatoes.

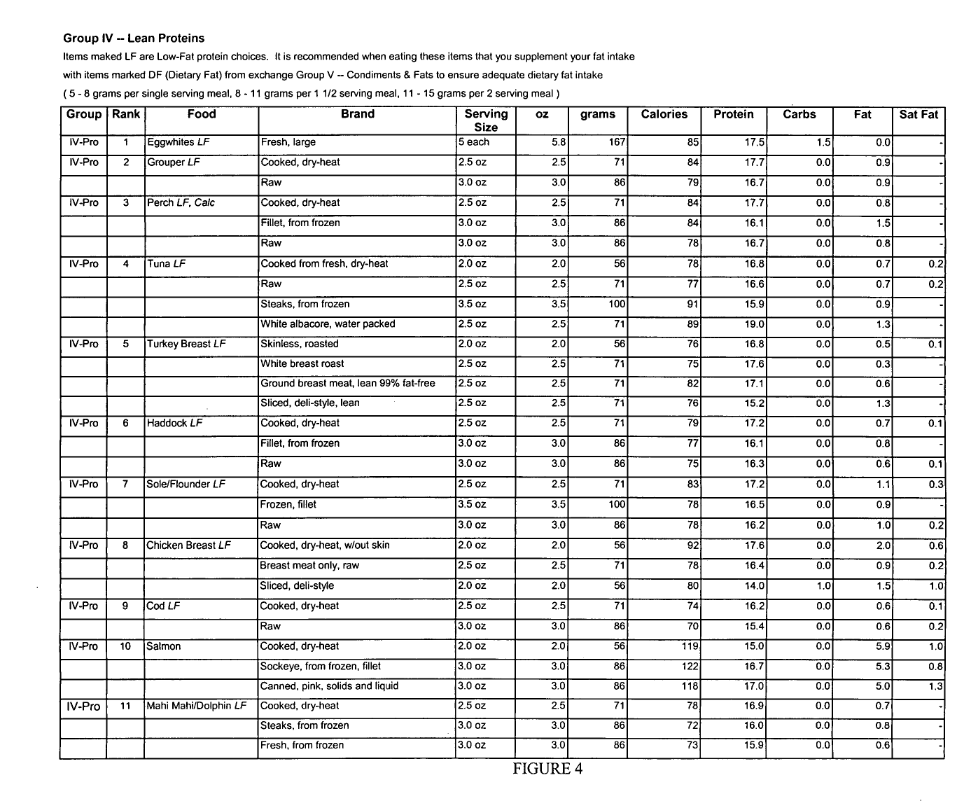

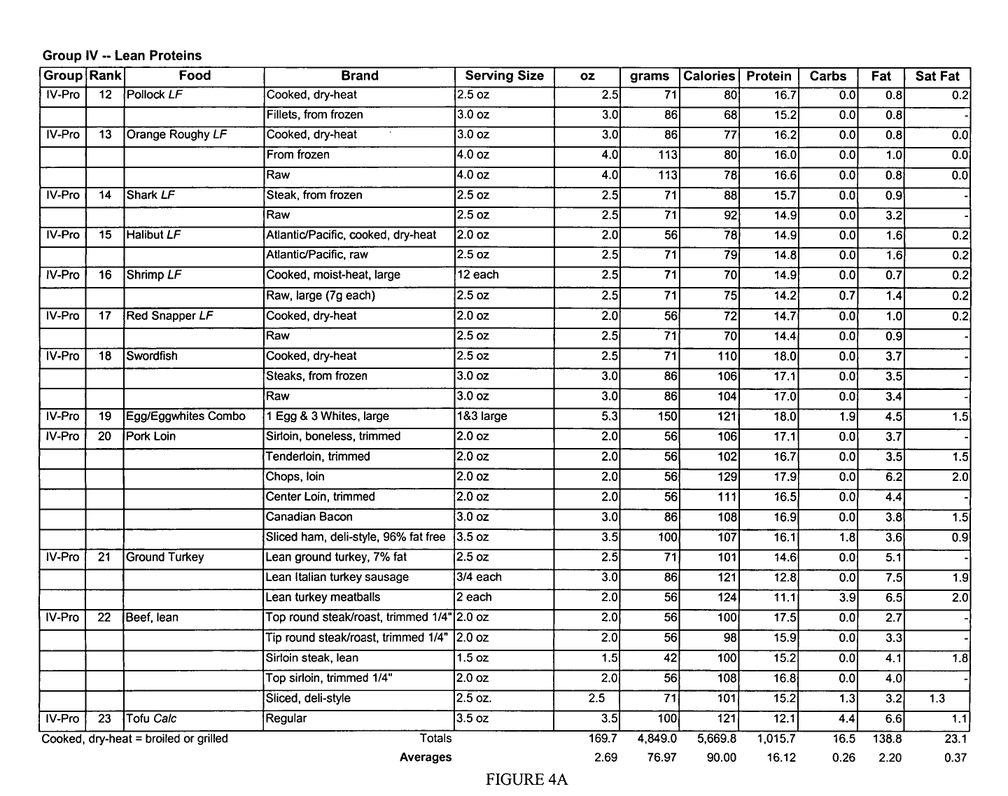

With reference to FIG. 4, lean proteins may be chosen from the group consisting of egg whites, grouper, perch, tuna, turkey breast, haddock, sole/flounder, mahi mahi/dolphin, pollock, chicken breasts, shrimp, cod, shark, halibut, orange roughy, red snapper, swordfish, egg/egg whites combo, pork loin, salmon, ground turkey, lean beef, and tofu.

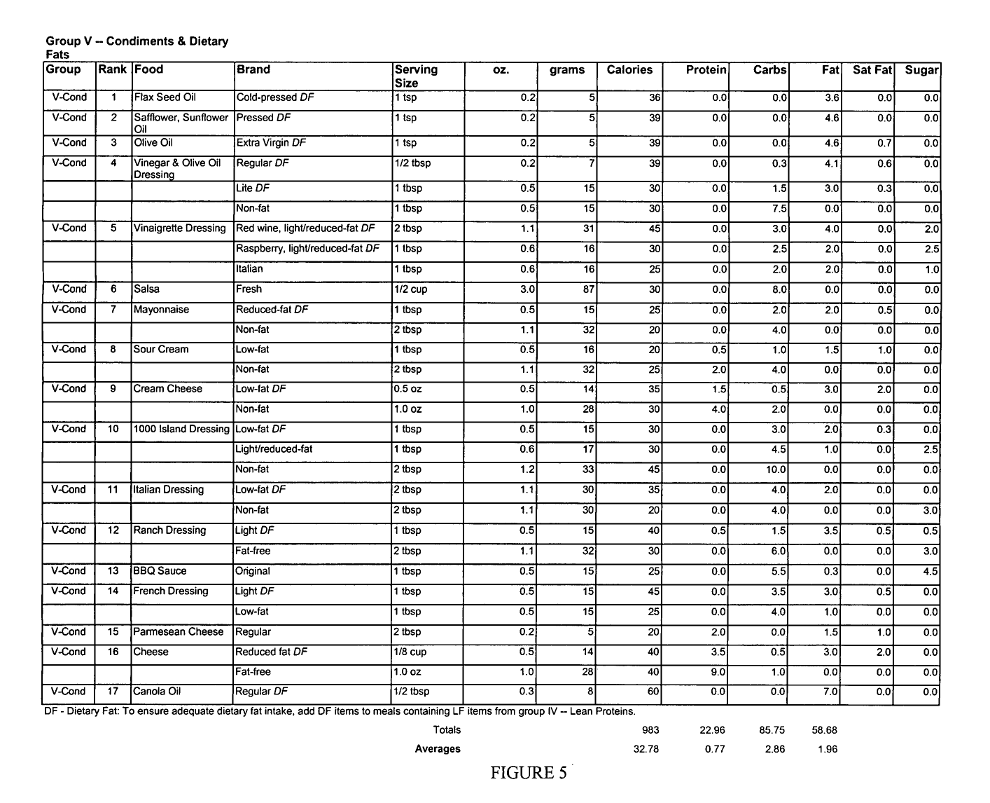

With reference to FIG. 5, condiments may be chosen from the group consisting of flax seed oil, safflower/sunflower, olive oil, vinegar and olive oil dressing, vinaigrette dressing, salsa, mayonnaise, sour cream, cream cheese, Thousand Island dressing, Italian dressing, ranch dressing, barbeque sauce, French dressing, parmesan cheese, cheese, and canola oil.

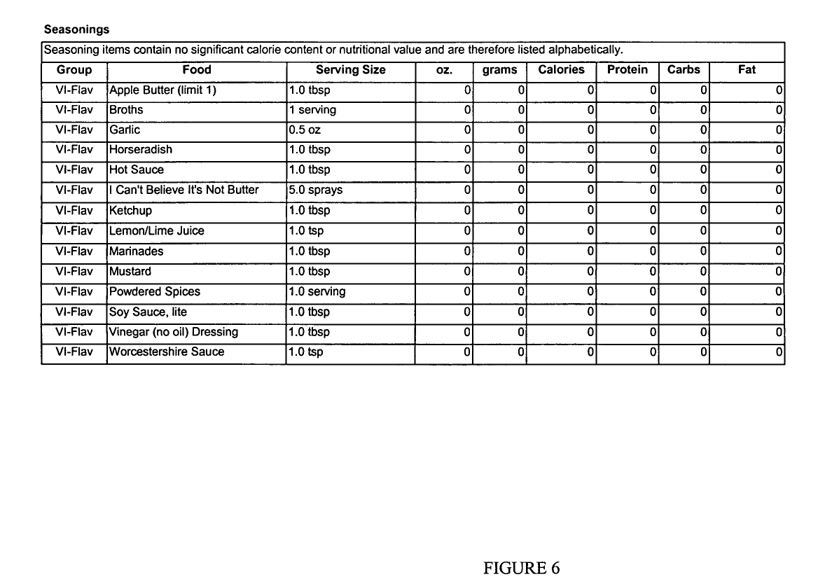

With reference to FIG. 6, seasonings may be chosen from the group consisting of apple butter, broths, garlic, horseradish, hot sauce, I Can't Believe It's Not Butter, ketchup, lemon, lime juice, marinades, mustard, powdered spices, light soy sauce, vinegar dressing, and Worcestershire sauce.

With reference to FIGS. 1-6, each of the foods chosen within a respective group should be limited by its serving size. The serving size results in a given number of calories with a certain ratio of protein, carbohydrates, fats, and fiber. The combination of servings from group one or group two, in conjunction with group three and group four will result in a 300 calorie meal to optimize weight loss and body composition for women and a 450 calorie meal for men.

Once a person is consistently eating five complete meals a day every 2½ to 3½ hours and getting the proper fat intake, a sixth complete meal may be added to increase the caloric intake to 1800 calories per day for women and 2700 calories a day for men. With respect to exercise, the present invention paired with 3-5 days of consistent and effect cardiovascular and weight resistant training may optimize results.

It is also contemplated to be within the scope of the invention to provide recipes and/or prepackaged foods already having the proper portion size with the requisite amount of calories and protein, carbohydrate, and fat ratios. Women can use the muffins, meal replacement bars to meet their caloric requirements by consuming one muffin or replacement bar for a 300 calorie meal or 1½ muffins or 1½ replacement bars for a 450 calorie meal. Men should consume 1½ muffins or meal replacement bars for a 450 calorie meal or 2 muffins or meal replacement bars for a 600 calorie meal. Muffins may be in the flavors, including but not limited to, apple cinnamon, banana-nutmeg, blueberry, cherry vanilla, and pumpkin. Further, the muffins may take any form chosen with sound judgment, including, but not limited to a cup shape or a bar shape.

An example of such recipes is as follows. The muffin recipes below include a ½ teaspoon of calcium powder, which is optional.

Blueberry Muffin/Bar

| | | | | | | | | Recipe for 6 | |
|---|---|---|---|---|---|---|---|---|---|
| | | | | | | | | Muffins | |
| | Qty | Qty | | | | | | | Qty |
| Item | Grams | oz. | Calories | Protein | Carbs | Fat | Fiber | Qty oz. | grams |
| Egg Whites | 50.00 | 1.75 | 29.75 | 6.13 | 0.53 | 0.00 | 0.00 | 10.50 | 300.00 |
| Whey Protein | 16.50 | 0.58 | 67.03 | 11.34 | 2.58 | 0.77 | 0.51 | 3.47 | 99.00 |
| Rolled Oats | 17.00 | 0.60 | 63.75 | 2.12 | 11.47 | 1.27 | 1.70 | 3.57 | 102.00 |
| Cornstarch | 3.50 | 0.12 | 13.02 | 0.00 | 3.04 | 0.00 | 0.00 | 0.49 | 14.00 |
| Ground Flax Seed | 3.00 | 0.11 | 13.73 | 0.69 | 0.92 | 1.03 | 0.92 | 0.63 | 18.00 |
| Almond Flour | 6.00 | 0.21 | 33.60 | 1.26 | 1.26 | 2.94 | 0.63 | 1.26 | 36.00 |
| Oat Bran | 5.00 | 0.18 | 18.75 | 0.88 | 3.13 | 0.37 | 0.75 | 1.05 | 30.00 |
| Apple Sauce, | 20.00 | 0.70 | 8.20 | 0.00 | 1.97 | 0.00 | 0.33 | 4.20 | 120.00 |
| unsweet | | | | | | | | | |
| Blueberries | 42.00 | 1.47 | 23.52 | 0.29 | 5.88 | 0.15 | 1.03 | 8.82 | 252.00 |
| Fructose powder | 8.50 | 0.30 | 31.83 | 0.00 | 8.50 | 0.00 | 0.00 | 1.79 | 51.00 |
| Baking Powder | 0.50 | 0.02 | ¼ tsp | | | | 1½ tsp | 0.11 | 7.00 |
| Xantham Gum | 0.25 | 0.01 | 0.25 | 0.00 | 0.01 | 0.00 | 0.01 | 0.04 | 1.00 |
| Salt | 1.00 | 0.04 | ⅛ tsp | | | | ¾ tsp | 0.21 | 4.00 |
| Calcium 500 mg | 1.50 | 0.05 | ½ tsp | | | | 3 tsp | 0.32 | 9.00 |
| 1 Muffin Totals | 173.25 | 6.06 | 303.43 | 22.71 | 39.27 | 6.54 | 5.87 | 35.77 | 1022.00 |
| | | | | 30% | 52% | 19% | | | |

Apple Cinnamon Muffin/Bar

| | | | | | | | | Recipe for | |
|---|---|---|---|---|---|---|---|---|---|
| | | | | | | | | 6 Muffins | |
| | Qty | Qty | | | | | | | Qty |
| Item | Grams | oz. | Calories | Protein | Carbs | Fat | Fiber | Qty oz. | grams |
| Egg Whites | 50.00 | 1.75 | 29.75 | 6.13 | 0.53 | 0.00 | 0.00 | 10.50 | 300.00 |
| Whey Protein | 17.00 | 0.60 | 69.06 | 11.69 | 2.65 | 0.80 | 0.53 | 3.57 | 102.00 |
| Rolled Oats | 16.00 | 0.56 | 60.00 | 2.00 | 10.80 | 1.20 | 1.60 | 3.36 | 96.00 |
| Cornstarch | 3.00 | 0.11 | 11.16 | 0.00 | 2.60 | 0.00 | 0.00 | 0.42 | 12.00 |
| Ground Flax Seed | 3.00 | 0.11 | 13.73 | 0.69 | 0.92 | 1.03 | 0.92 | 0.63 | 18.00 |
| Almond Flour | 6.00 | 0.21 | 33.60 | 1.26 | 1.26 | 2.94 | 0.63 | 1.26 | 36.00 |
| Oat Bran | 3.00 | 0.11 | 11.25 | 0.53 | 1.88 | 0.22 | 0.45 | 0.63 | 18.00 |
| Apple sauce | 30.00 | 1.05 | 12.30 | 0.00 | 2.95 | 0.00 | 0.49 | 6.30 | 180.00 |
| Apples | 56.00 | 1.96 | 32.67 | 0.25 | 8.35 | 0.25 | 1.31 | 11.76 | 336.00 |
| Fructose | 8.00 | 0.28 | 29.96 | 0.00 | 8.00 | 0.00 | 0.00 | 1.68 | 48.00 |
| Apple Pie Spice | 2.00 | 0.07 | ⅔ tsp | | | | 4 tsp | 0.42 | 12.00 |
| Cinnamon | 1.86 | 0.07 | ⅔ tsp | | | | 4 tsp | 0.39 | 11.16 |
| Baking Powder | 0.50 | 0.02 | ¼ tsp | | | | 1½ tsp | 0.11 | 3.00 |
| Salt | 1.00 | 0.04 | ⅛ tsp | | | | ¾ tsp | 0.21 | 6.00 |
| Baking Soda | 0.17 | 0.01 | 1/16 tsp | | | | ⅜ tsp | 0.04 | 1.02 |
| Calcium 500 mg | 1.50 | 0.05 | ½ tsp | | | | 3 tsp | 0.32 | 9.00 |
| 1 Muffin Totals | 197.53 | 6.91 | 303.49 | 22.54 | 39.93 | 6.45 | 5.93 | 40.11 | 1146.00 |
| | | | | 30% | 53% | 19% | | | |

Banana Nutmeg Muffin/Bar

| | | | | | | | | Recipe for | |
|---|---|---|---|---|---|---|---|---|---|
| | Qty | | | | | | | 6 Muffins | |
| Item | Grams | Qty oz. | Calories | Protein | Carbs | Fat | Fiber | Qty oz. | Qty grams |
| Egg Whites | 50.00 | 1.75 | 29.75 | 6.13 | 0.53 | 0.00 | 0.00 | 10.50 | 300.00 |
| Whey Protein | 16.50 | 0.58 | 67.03 | 11.34 | 2.58 | 0.77 | 0.51 | 3.47 | 99.00 |
| Rolled Oats | 16.50 | 0.58 | 61.87 | 2.06 | 11.13 | 1.24 | 1.65 | 3.47 | 99.00 |
| Cornstarch | 2.50 | 0.09 | 9.30 | 0.00 | 2.17 | 0.00 | 0.00 | 0.35 | 10.00 |
| Ground Flax Seed | 3.00 | 0.11 | 13.73 | 0.69 | 0.92 | 1.03 | 0.92 | 0.63 | 18.00 |
| Almond Flour | 6.00 | 0.21 | 33.60 | 1.26 | 1.26 | 2.94 | 0.63 | 1.26 | 36.00 |
| Oat Bran | 3.00 | 0.11 | 11.25 | 0.53 | 1.88 | 0.22 | 0.45 | 0.63 | 18.00 |
| Bananas | 58.00 | 2.03 | 52.78 | 0.61 | 13.40 | 0.20 | 1.02 | 12.18 | 348.00 |
| Fructose | 7.00 | 0.25 | 26.22 | 0.00 | 7.00 | 0.00 | 0.00 | 1.47 | 42.00 |
| Vanilla | 2.17 | 0.08 | ½ tsp | | | | 3 tsp | 0.46 | 13.02 |
| Nutmeg | 1.17 | 0.04 | ¼ tsp | | | | 1½ tsp | 0.25 | 7.02 |
| Allspice | 1.17 | 0.04 | ¼ tsp | | | | 1½ tsp | 0.25 | 7.02 |
| Baking Powder | 0.50 | 0.02 | ¼ tsp | | | | 1½ tsp | 0.11 | 3.00 |
| Salt | 1.00 | 0.04 | ⅛ tsp | | | | ¾ tsp | 0.21 | 6.00 |
| Baking Soda | 0.17 | 0.01 | 1/16 tsp | | | | ⅜ tsp | 0.04 | 1.02 |
| Calcium 500 mg | 1.50 | 0.05 | ½ tsp | | | | 3 tsp | 0.32 | 9.00 |
| 1 Muffin Total | 168.68 | 5.90 | 305.53 | 22.61 | 40.85 | 6.41 | 5.17 | 33.95 | 970.00 |
| | | | | 30% | 53% | 19% | | | |

Cherry Vanilla Muffin/Bar

| | | | | | | | | Recipe for 6 | |
|---|---|---|---|---|---|---|---|---|---|
| | | | | | | | | Muffins | |
| | Qty | | | | | | | | Qty |
| Item | Grams | Qty oz. | Calories | Protein | Carbs | Fat | Fiber | Qty oz. | grams |
| Egg Whites | 50.00 | 1.75 | 29.75 | 6.13 | 0.53 | 0.00 | 0.00 | 10.50 | 300.00 |
| Whey Protein | 16.50 | 0.58 | 67.03 | 11.34 | 2.58 | 0.77 | 0.51 | 3.47 | 99.00 |
| Rolled Oats | 16.00 | 0.56 | 60.00 | 2.00 | 10.80 | 1.20 | 1.60 | 3.36 | 96.00 |
| Cornstarch | 3.00 | 0.11 | 11.16 | 0.00 | 2.60 | 0.00 | 0.00 | 0.42 | 12.00 |
| Ground Flax Seed | 3.00 | 0.11 | 13.73 | 0.69 | 0.92 | 1.03 | 0.92 | 0.63 | 18.00 |
| Almond Flour | 6.00 | 0.21 | 33.60 | 1.26 | 1.26 | 2.94 | 0.63 | 1.26 | 36.00 |
| Oat Bran | 5.00 | 0.18 | 18.75 | 0.88 | 3.13 | 0.37 | 0.75 | 1.05 | 30.00 |
| Apple sauce | 0.00 | 0.00 | 0.00 | 0.00 | 0.00 | 0.00 | 0.00 | 0.00 | 0.00 |
| Yogurt | 15.00 | 0.53 | 12.50 | 0.59 | 2.24 | 0.13 | 0.20 | 3.15 | 90.00 |
| Cherries | 58.00 | 2.03 | 35.99 | 0.00 | 8.79 | 0.00 | 0.41 | 12.18 | 348.00 |
| Fructose | 6.00 | 0.21 | 22.47 | 0.00 | 6.00 | 0.00 | 0.00 | 1.26 | 36.00 |
| Lime Juice | 3.35 | 0.12 | ½ tsp | | | | | 0.70 | 3 tsp |
| Vanilla | 2.17 | 0.08 | 1 tsp | | | | | 0.46 | 6 tsp |
| Baking Powder | 0.50 | 0.02 | ¼ tsp | | | | | 0.11 | 1½ tsp |
| Salt | 1.00 | 0.04 | ⅛ tsp | | | | | 0.21 | ¾ tsp |
| Baking Soda | 0.17 | 0.01 | 1/16 tsp | | | | | 0.04 | ⅜ tsp |
| Calcium 250 mg | 0.75 | 0.03 | ¼ tsp | | | | 1.5 tsp | 0.16 | 4.50 |
| 1 Muffin Totals | 185.69 | 6.50 | 304.99 | 22.88 | 38.83 | 6.45 | 5.01 | 37.28 | 1065.00 |
| | | | | 30% | 51% | 19% | | | |

Pumpkin Muffin/Bar

| | | | | | | | | Recipe for 6 | |
|---|---|---|---|---|---|---|---|---|---|
| | Qty | Qty | | | | | | Muffins | |
| Item | Grams | oz. | Calories | Protein | Carbs | Fat | Fiber | Qty oz. | Qty grams |
| Egg Whites | 50.00 | 1.75 | 29.75 | 6.13 | 0.53 | 0.00 | 0.00 | 10.50 | 300.00 |
| Whey Protein | 16.50 | 0.58 | 67.03 | 11.34 | 2.58 | 0.77 | 0.51 | 3.47 | 99.00 |
| Rolled Oats | 16.50 | 0.58 | 61.87 | 2.06 | 11.13 | 1.24 | 1.65 | 3.47 | 99.00 |
| Cornstarch | 3.00 | 0.11 | 11.16 | 0.00 | 2.60 | 0.00 | 0.00 | 0.42 | 12.00 |
| Ground Flax Seed | 3.00 | 0.11 | 13.73 | 0.69 | 0.92 | 1.03 | 0.92 | 0.63 | 18.00 |
| Almond Flour | 6.00 | 0.21 | 33.60 | 1.26 | 1.26 | 2.94 | 0.63 | 1.26 | 36.00 |
| Oat Bran | 2.50 | 0.09 | 9.37 | 0.44 | 1.56 | 0.19 | 0.37 | 0.53 | 15.00 |
| Pumpkin | 115.00 | 4.03 | 47.13 | 0.93 | 9.42 | 0.00 | 3.78 | 24.15 | 690.00 |
| Fructose | 7.50 | 0.26 | 28.09 | 0.00 | 7.50 | 0.00 | 0.00 | 1.58 | 45.00 |
| Honey | 1.50 | 0.05 | 4.26 | 0.00 | 1.21 | 0.00 | 0.00 | 0.32 | 9.00 |
| Pumpkin Pie | 2.00 | 0.07 | ⅔ tsp | | | | 4 tsp | 0.42 | 12.00 |
| Spice | | | | | | | | | |
| Cinnamon | 1.86 | 0.07 | ⅔ tsp | | | | 4 tsp | 0.39 | 11.16 |
| Baking Powder | 0.50 | 0.02 | ¼ tsp | | | | 1½ tsp | 0.11 | 3.00 |
| Salt | 1.00 | 0.04 | ⅛ tsp | | | | ¾ tsp | 0.21 | 6.00 |
| Baking Soda | 0.17 | 0.01 | 1/16 tsp | | | | ⅜ tsp | 0.04 | 1.02 |
| Calcium 500 mg | 1.50 | 0.05 | ½ tsp | | | | 3 tsp | 0.32 | 9.00 |
| 1 Muffin Totals | 228.53 | 8.00 | 306.00 | 22.84 | 38.70 | 6.17 | 7.87 | 45.99 | 1314.00 |
| | | | | 30% | 51% | 18% | | | |

It is yet another object of the present invention to provide recipes for fruit yogurts, turkey chili, turkey chili with pasta, chicken potato soup, chicken potato and pasta soup, chicken whole wheat burrito, whole wheat pizza, and whole wheat pita pizza. Such recipes are as follows:

Chicken Potato Soup

| | | | | | | | | | 8-1 serving | |
|---|---|---|---|---|---|---|---|---|---|---|
| | Qty | | | | | | | | Measurements | |
| Item | Grams | Qty oz. | Cal | Pro | Carbs | Fat | Fiber | Qty oz. | Qty grams | Total Qty |
| Chicken Breast | 56.00 | 2.2 | 101.20 | 19.36 | 0.00 | 2.20 | 0.66 | 17.60 | 448.00 | 1 lb 2 oz |
| Potato - boiled | 142.00 | 4.5 | 124.62 | 3.38 | 27.43 | 0.17 | 2.94 | 36.00 | 1136.00 | 2 lb 4 oz |
| Carrot | 85.00 | 2.5 | 33.00 | 0.80 | 6.70 | 0.80 | 0.80 | 20.00 | 680.00 | 1 lb 4 oz |
| Celery | 56.00 | 2.0 | 10.27 | 0.47 | 1.87 | 0.09 | 0.93 | 15.68 | 448.00 | 1 lb |
| Onion | 40.00 | 1.4 | 15.50 | 0.50 | 3.50 | 0.10 | 0.75 | 11.20 | 320.00 | 2 cups |
| Olive oil | 2.50 | 0.1 | 17.06 | 0.00 | 0.00 | 2.01 | 0.31 | 0.70 | 20.00 | 3½ tsp |
| Water | 2¼ cups | | | | | | | | | 18 cups |
| 2 Chicken | | | | | | | | | | 16 cubes |
| Boullion Cubes | 1 Serving | Totals | 301.64 | 24.50 | 39.50 | 5.38 | 6.39 | | | |
| | | | | 32% | 52% | 16% | | | | |

Chicken Potato & Pasta Soup

| | | | | | | | | | 8-1 serving | |
|---|---|---|---|---|---|---|---|---|---|---|
| | Qty | | | | | | | | Measurements | |
| Item | Grams | Qty oz. | Cal | Pro | Carbs | Fat | Fiber | Qty oz. | Qty grams | Total Qty |
| Chicken Breast | 56.00 | 2.2 | 98.90 | 18.92 | 0.00 | 2.15 | 0.65 | 17.20 | 448.00 | 1 lb 2 oz |
| Potato - boiled | 71.00 | 2.0 | 55.38 | 1.50 | 12.19 | 0.08 | 1.31 | 16.00 | 568.00 | 1 lb |
| Pasta - durum | 42.00 | 1.5 | 75.66 | 2.51 | 14.79 | 0.35 | 0.74 | 11.76 | 336.00 | 12 oz -- |
| semolina | | | | | | | | | | 2.65 cups |
| Carrot | 85.00 | 2.5 | 33.00 | 0.80 | 6.70 | 0.80 | 0.80 | 20.00 | 680.00 | 1 lb 4 oz |
| Celery | 56.00 | 2.0 | 10.27 | 0.47 | 1.87 | 0.09 | 0.93 | 15.68 | 448.00 | 1 lb |
| Onion | 40.00 | 1.4 | 15.50 | 0.50 | 3.50 | 0.10 | 0.75 | 11.20 | 320.00 | 2 cups |
| Olive oil | 2.50 | 0.1 | 17.06 | 0.00 | 0.00 | 2.01 | 0.31 | 0.70 | 20.00 | 3½ tsp |
| Water | 2¼ cups | | | | | | | | | 18 cups |
| 2 Chicken | | | | | | | | | | 16 cubes |
| Boullion Cubes | 1 Serving | Totals | 305.78 | 24.69 | 39.05 | 5.58 | 5.48 | | | |
| | | | | 32% | 51% | 16% | | | | |

Turkey Chili

| | | | | | | | | | 8-1 serving |
|---|---|---|---|---|---|---|---|---|---|
| Ingredients | ounces | grams | Cal | Pro | Carbs | Fat | Fiber | Qty Oz. | Measurements |
| Lean Ground | 2.00 | 56.70 | 80.00 | 11.50 | 0.00 | 4.00 | 0.00 | 16.00 | 1 lb Cooked |
| Turkey | | | | | | | | | |
| Kidney Beans | 5.90 | 167.27 | 167.44 | 10.33 | 27.02 | 1.30 | 9.03 | 47.20 | 5 cans @ 9.5 oz |
| Tomato Sauce | 2.81 | 79.66 | 19.33 | 1.29 | 3.88 | 0.00 | 1.29 | 22.48 | 1½ cans @ 15 oz |
| Diced Tomatoes | 3.62 | 102.63 | 16.94 | 0.83 | 4.24 | 0.00 | 0.83 | 28.96 | 2 cans @ 14.5 oz |
| Onion | 2.00 | 56.70 | 22.00 | 0.60 | 4.80 | 0.20 | 1.00 | 16.00 | 2 each @ 8 oz |
| Chili Seasoning | 0.24 | 6.80 | 1.12 | 0.06 | 0.28 | 0.00 | 0.06 | 1.92 | 1½ packs @ 1.25 oz |
| | | 0.00 | 0.00 | 0.00 | 0.00 | 0.00 | 0.00 | 0.00 | |
| | 1 Serving | Totals | 306.84 | 24.61 | 40.22 | 5.50 | 12.21 | | |
| | | | | 32% | 52% | 16% | | | |

Turkey Chili & Pasta

| | | | | | | | | | 8-1 Serving |
|---|---|---|---|---|---|---|---|---|---|
| Item | ounces | grams | Calories | Protein | Carbs | Fat | Fiber | Qty Oz. | Measurements |
| Lean Ground | 2.50 | 70.88 | 100.00 | 14.38 | 0.00 | 5.00 | 0.00 | 20.00 | 1 lb, 4 oz cooked |
| Turkey | | | | | | | | | |
| Pasta | 1.50 | 42.53 | 78.29 | 2.61 | 15.29 | 0.38 | 0.75 | 12.00 | ¾ lb (16 oz), dry |
| Kidney beans | 2.38 | 67.47 | 67.54 | 4.17 | 10.90 | 0.52 | 3.64 | 19.04 | 2 cans @ 9.5 oz |
| Tomato Sauce | 3.75 | 106.31 | 25.80 | 1.73 | 5.18 | 0.00 | 1.73 | 30.00 | 2 cans @15 oz |
| Diced Tomatoes | 3.63 | 102.91 | 16.99 | 0.83 | 4.25 | 0.00 | 0.83 | 29.04 | 2 cans @14.5 oz |
| Green Peppers | 0.75 | 21.26 | 4.69 | 0.17 | 1.13 | 0.23 | 0.60 | 6.00 | 1 ea @ 6 oz |
| Onion | 0.75 | 21.26 | 8.25 | 0.23 | 1.80 | 0.08 | 0.38 | 6.00 | 1 ea |
| Chili Seasoning | 0.24 | 6.80 | 1.12 | 0.06 | 0.28 | 0.00 | 0.06 | 1.92 | 1½ packs |
| | | 0.00 | 0.00 | 0.00 | 0.00 | 0.00 | 0.00 | 0.00 | |
| | 1 Serving | Total | 302.68 | 24.16 | 38.81 | 6.20 | 7.98 | | |
| | | | | 32% | 51% | 18% | | | |

Chicken Whole Wheat Burrito

| | | | | | | | | Recipe - 8 Burritos | |
|---|---|---|---|---|---|---|---|---|---|
| | Qty | | | | | | | | Qty |
| Item | Grams | Qty oz. | Calories | Protein | Carbs | Fat | Fiber | Qty oz. | grams |
| Chicken Breast, diced 1 | 43 | 1.50 | 70.76 | 13.46 | 0.00 | 1.50 | 0.00 | 12.00 | 255.15 |
| Tortilla, whole wheat, | 65 | 2.30 | 149.87 | 6.92 | 31.13 | 5.76 | 1.15 | 18.40 | 391.23 |
| med | | | | | | | | | |
| Rice, converted, cooked | 35 | 1.25 | 33.73 | 0.79 | 7.54 | 0.00 | 0.00 | 10.00 | 212.63 |
| Blackbeans, cooked | 28 | 1.00 | 24.18 | 1.54 | 4.18 | 0.22 | 1.54 | 8.00 | 170.10 |
| Tomato, diced | 99 | 3.50 | 21.11 | 1.11 | 4.44 | 0.28 | 1.67 | 28.00 | 595.35 |
| 1 Serving Totals | 271 | 9.55 | 299.66 | 23.82 | 47.29 | 7.76 | 4.36 | 76.40 | 1624.46 |
| | | | | 32% | 63% | 23% | | | |

1 - Chicken breast can be substituted with 1.75 oz. of Lean Sirloin Beef, or 3.0 oz Mahi Mahi fish.

Whole Wheat Pita Pizza

| | Qty | | | | | | | Recipe - 8 Pizzas | |
|---|---|---|---|---|---|---|---|---|---|
| Item | Grams | Qty oz. | Calories | Protein | Carbs | Fat | Fiber | Qty oz. | Qty grams |
| Chicken breast, cubed 1 | 42.53 | 1.50 | 70.76 | 13.46 | 0.00 | 1.50 | 0.00 | 12.00 | 255.15 |
| Pita Bread, med | 63.79 | 2.25 | 150.46 | 5.47 | 32.83 | 0.68 | 4.10 | 18.00 | 382.73 |
| Pasta Sauce, low-fat | 56.70 | 2.00 | 23.04 | 0.92 | 4.15 | 0.46 | 0.92 | 16.00 | 340.20 |
| Mushrooms, sliced 2 | 63.79 | 2.25 | 16.53 | 1.84 | 3.67 | 0.92 | 1.84 | 18.00 | 382.73 |
| Cheese, reduced-fat | 14.18 | 0.50 | 40.31 | 3.53 | 0.50 | 3.02 | 0.00 | 4.00 | 85.05 |
| | 240.98 | 8.50 | 301.10 | 25.21 | 41.15 | 6.58 | 6.86 | 68.00 | 1445.85 |
| | | | | 33% | 55% | 20% | | | |

1 - Chicken breast can be substituted with 2.0 oz. of Lean turkey sausage, 3.0 oz. Lean ground turkey, or 3.0 oz. of Canadian Bacon.
2 - Mushrooms can be substituted with 1.4 oz. of Onion, 1.8 oz. of Sweet Peppers, or 2.4 oz. tomato.

Whole Wheat Pizza

| | | | | | | | | | | | 5 Single |
|---|---|---|---|---|---|---|---|---|---|---|---|
| | | | | | | | | | | | Serving |
| | | Qty | Qty | | | | | | Qty | Qty | Slices |
| Item | Size | Grams | oz. | Calories | Protein | Carbs | Fat | Fiber | oz. | grams | Item |
| Mushrooms | 1 ea | 11.34 | 0.40 | 2.72 | 0.41 | 0.41 | 0.00 | 0.14 | 2.00 | 56.70 | 5 |
| | | | | | | | | | | | mushrooms |
| Sweet Pepper | 1/10 | 17.01 | 0.60 | 2.73 | 0.11 | 0.55 | 0.01 | 0.22 | 3.00 | 85.05 | ½ large |
| | large | | | | | | | | | | |
| Onion | 1/10 | 17.01 | 0.60 | 6.95 | 0.12 | 1.85 | 0.02 | 0.35 | 3.00 | 85.05 | ½ |
| | med | | | | | | | | | | medium |
| Pasta Sauce | >⅕ cup | 51.03 | 1.80 | 27.07 | 0.77 | 4.25 | 0.97 | 1.55 | 9.00 | 255.15 | 1 cup |
| Wheat Pizza Crust | ⅕ shell | 56.70 | 2.00 | 150.38 | 7.02 | 27.07 | 3.01 | 4.01 | 10.00 | 283.50 | 1 large |
| | | | | | | | | | | | Shell |
| Lean Turkey | ½ link | 56.70 | 2.00 | 82.05 | 8.72 | 0.00 | 5.13 | 0.00 | 10.00 | 283.50 | 2 ½ links |
| Sausage 1 | | | | | | | | | | | |
| Cheese, Mozz, Fat | ⅕ cup | 11.20 | 0.80 | 36.28 | 6.45 | 1.61 | 0.00 | 0.00 | 4.00 | 56.00 | 1 cup |
| Free | | | | | | | | | | | |
| 1 Serving Totals | | | | 308.18 | 23.59 | 35.74 | 9.14 | 6.26 | | | |
| | | | | | 31% | 46% | 27% | | | | |

1 Lean Turkey Sausage can be substituted with 2 oz of Ground Turkey or 1.5 oz of Chicken Breast

Strawberry Whey Yogurt

| | | Qty | | | | | | |
|---|---|---|---|---|---|---|---|---|
| Food | Size | Grams | Qty oz. | Calories | Protein | Carbs | Fat | Fiber |
| Whey protein | ½ scoop + | 19 | 0.67 | 77.19 | 13.06 | 2.97 | 0.89 | 0.59 |
| Yogurt, low-fat | 1 cup | 220 | 7.70 | 184.14 | 8.72 | 32.95 | 1.94 | 2.91 |
| Vanilla | | | | | | | | |
| Strawberries 1 | ⅔ cup | 99 | 3.47 | 29.90 | 0.66 | 6.91 | 0.33 | 2.66 |
| Fructose | 1 tsp | 4 | 0.14 | 15.10 | 0.00 | 4.03 | 0.00 | 0.00 |
| 1 Serving Total | | 342.00 | 11.97 | 306.32 | 22.45 | 46.86 | 3.16 | 6.16 |
| | | | | | 29% | 61% | 9% | |

Strawberries can be substitued with the following fruits:

| Fruit | grams |
|---|---|
| Apples | 50 |
| Blueberries | 54 |
| Cherries | 45 |
| Peach | 70 |
| Pineapple | 54 |
| Raspberries | 61 |

With the above-described recipes, heating and cooking are those temperatures and methods chosen in accordance with sound engineering judgment and are well known in the art. For example, the muffins/bars may be baked at an oven temperature of about 350 degrees until a knife can be inserted and cleanly removed. Further, the soups and chili recipes may be cooked over a stove top or in a crock pot at a temperature to thoroughly cook the ingredients without burning.

Studies were conducted using the Top 100 Food List, as shown in FIGS. 1-6. In these studies the optimal ranges of protein, carbohydrates, and fat were determined. By following the present invention, 335 women lost 3,337.50 pounds of body fat while increasing their lean muscle mass by 671.00 pounds. And 208 men lost 2,034.32 pounds of body fat while increasing their lean muscle mass by 412.07 pounds.

With respect to children ages 4 to 18, proper and adequate nutrition is never more important than during the growing years. Children need a consistent supply of proper and balanced nutrients to grow and function at an optimal level. Consistently providing children with the right type and amount of nutrients is also vital to increasing their daily energy levels so they can achieve and maintain ideal health, fitness, and body composition levels later in life. Not all foods, however, have the same effect on a child's metabolism.

FIGS. 1 to 6 show the foods listed in the Top 100 Food System for children. Foods are ranked according to their nutritional value and the effects they have on the body during and after digestion. The number one ranking represents the top food choice for each of the specific food groups. All other choices are listed in declining order. The quantities listed for each of the Top 100 Food items represents one serving size.

Nutritional requirements for children are affected by a multitude of factors. The Top 100 Foods System for Kids is meant to provide general nutrition guidelines for children ages 4 to 18. For more specific information regarding a child's nutritional needs, a parent or guardian must consult with their Family Physician, Pediatrician, or a Registered Dietician.

It is not required that children only consume foods that are listed in the Top 100 Food System. However, consistently consuming a balanced variety of the listed foods in the appropriate quantities can produce more favorable metabolic reactions in helping to positively change body composition and improve daily energy levels. Paired with consistent and appropriate daily activity, the Top 100 Foods System can help give a family the energy needed to look great and feel even better so they can get the most out of life.

It is recommended that children consume at least two servings daily of low-fat dairy products to ensure adequate daily calcium intake. These dairy products should be consumed in addition to—not in place of—the following nutritional guidelines. Items marked Calc on the Top 100 Food System represent foods that are excellent sources of calcium. Calc items can also be consumed to help meet the daily calcium requirements. A calcium/magnesium supplement with vitamin D can also be used to meet daily calcium needs.

The Top 100 Food system will increase the fiber consumption of most people. For this reason it is recommended that 50-64 oz. of plain water per day be consumed daily. Adequate water consumption not only improves the efficiency of the fat-burning and body-shaping process, it also aids in the digestion, assimilation and elimination of dietary fiber. If significant gastrointestinal stress develops, discontinue the program and consult your physician.

Any nutrition plan will not be effective by itself. The nutrition plan outlined within is safe and effective for use only if coupled with consistent and age appropriate exercise and physical activity.

The following are nutrition suggestions for selected age groups.

Children (4-8 years of age)—To ensure adequate and consistent nutrient intake, 4-8 year olds should eat at least 5 balanced meals per day; each meal every 2½ to 3½ hours. Each balanced meal should consist of:
1 serving of Vegetables (Group I) or Fruit (Group II) and
½-1 serving of Complex Carbohydrate (Group III), and
Less than ½ serving of Lean Protein (Group IV).

Five balanced meals per day with 1 serving of Vegetables or Fruit, ½-1 serving of Complex Carbohydrates, and less than ½ serving of Lean Protein averages up to 250 calories per meal at 15-20% protein, 55-60% carbohydrate, and 20-25% fat with 5-7 grams of fiber; for a total of up to 1250 calories per day.

If they are consistently eating 5 balanced meals per day, every 2½ to 3½ hours, and getting in at least 5-7 grams of fat per meal; and they feel like they want or need more food, then add a 6th balanced meal. This will increase their caloric intake to approximately 1500 calories per day. From there you can systematically increase their caloric intake as needed by increasing the serving size of fruits and vegetables to 1½ servings, complex carbohydrates to 1 serving and lean proteins to ½ servings for any of their meals.

Children (9-13 years of age)—To ensure adequate and consistent nutrient intake, 9-13 year olds should eat at least 5 balanced meals per day; each meal every 2½ to 3½ hours. Each balanced meal should consist of:
1 serving of Vegetables (Group I) or Fruit (Group II) and
1 serving of Complex Carbohydrate (Group III), and
½ serving of Lean Protein (Group IV).

Five balanced meals per day with 1 serving each of Vegetables or Fruit, 1 serving of Complex Carbohydrates, and ½ serving of Lean Protein averages 265 calories per meal at 15-20% protein, 55-60% carbohydrate, and 20-25% fat with 6-7 grams of fiber; for a total of 1350 calories per day.

If they are consistently eating 5 balanced meals per day, every 2½ to 3½ hours, and getting in at least 6-8 grams of fat per meal; and they feel like they want or need more food, then add a 6th balanced meal. This will increase their caloric intake to approximately 1600 calories per day. From there you can systematically increase their caloric intake as needed by increasing the serving size of fruits, vegetables and complex carbohydrates to 1½ servings and lean proteins to ½-¾ serving for any of their meals.

Children (14-18 years of age)—To ensure adequate and consistent nutrient intake, 14-18 year olds should eat at least 5 balanced meals per day; each meal every 2½ to 3½ hours. Each balanced meal should consist of:
1 serving of Vegetables (Group I) or Fruit (Group II) and
1 serving of Complex Carbohydrate (Group III), and
½-¾ servings of Lean Protein (Group IV).

Five balanced meals per day with 1 serving each of Vegetables or Fruit, 1 serving of Complex Carbohydrates, and ½-¾ servings of Lean Protein averages 275 calories per meal at 20-25% protein, 50-55% carbohydrate, and 20-25% fat with 6-7 grams of fiber; for a total of 1400 calories per day.

If they are consistently eating 5 balanced meals per day, every 2½ to 3½ hours, and getting in at least 6-8 grams of fat per meal; and they feel like they want or need more food, then add a 6th balanced meal. This will increase their caloric intake to approximately 1700 calories per day. From there you can systematically increase their caloric intake as needed by increasing the serving size of fruits, vegetables and complex carbohydrates to 1½ servings and lean proteins to ¾-1 serving for any of their meals.

The Top 100 Food System is for educational and informative purposes of healthy individuals only and is not intended as medical or professional advice. No health claims are made for these nutritional guidelines. Always consult your physician or health care professional before making any changes to your diet, nutrition, or exercise program.

In another embodiment of the present invention, meals and foods described herein may be readily made available at a point of purchase location, such as a grocery store or a stand alone store specializing in selling foods and meals as described herein.

The preferred embodiments have been described, hereinabove. It will be apparent to those skilled in the art that the above methods may incorporate changes and modifications without departing from the general scope of this invention. It is intended to include all such modifications and alterations in so far as they come within the scope of the appended claims or the equivalents thereof.

Having thus described the invention, it is now claimed:

1. A method for managing weight, comprising the steps of:
eating a meal at least five times a day with one serving of a vegetable or a fruit, a complex carbohydrate, and a lean protein for a predetermined number of calories per meal at ranges of 25-35% protein, 50-55% carbohydrate, 15-20% fat with a predetermined range of grams of fiber;
losing weight; and
optimizing body composition.

2. The method of claim 1, wherein said predetermined number of calories for women is about 300 calories and said predetermined range of grams of fiber is about six to seven grams.

3. The method of claim 1, wherein said predetermined number of calories for men is about 450 calories and said predetermined range of grams of fiber is about nine to ten grams of fiber.

4. The method of claim 1, wherein said step of eating a meal at least five times a day further comprises the step of eating one food that includes 25-35% protein, 50-55% carbohydrate, 15-20% fat.

5. The method of claim 4, wherein said one food is selected from the group a muffin, fruit yogurt, chicken potato soup, chicken potato & pasta soup, turkey chili, turkey chili & pasta, chicken whole wheat burrito, whole wheat pizza, or whole wheat pita pizza.

6. The method of claim 5, wherein said muffin is a pumpkin muffin comprising about 50 grams of egg whites, about 16.5 grams of whey protein, about 16.5 grams of rolled oats, about 3 grams of cornstarch, about 3 grams ground flax seed, about 6 grams of almond flour, about 2.5 grams of oat bran, about 115 grams of pumpkin, about 7.5 grams of fructose, about 1.5 grams of honey, about ⅔ teaspoon of pumpkin pie spice, about ⅔ teaspoon of cinnamon, about ¼ teaspoon of baking powder, about ⅛ teaspoon salt, and about 1/16 teaspoon of baking soda, so as to result in about one serving.

7. The method of claim 5, wherein said muffin is an apple cinnamon muffin, comprising about 50 grams of egg whites, about 17 grams of whey protein, about 16 grams of rolled oats, about 3 grams of cornstarch, about 3 grams of ground flax seed, about 6 grams of almond flour, about 3 grams of oat bran, about 30 grams of apple sauce, about 56 grams of apples, about 8 grams of fructose, about ⅔ teaspoon of apple pie spice, about ⅔ teaspoon of cinnamon, about ¼ teaspoon of baking powder, about ⅛ teaspoon of salt, and about 1/16 teaspoon of baking soda, so as to result in about one serving.

8. The method of claim 5, wherein said muffin is a banana-nutmeg muffin comprising about 50 grams of egg whites, about 16.5 grams of whey protein, about 16.5 grams of rolled oats, about 2.5 grams of cornstarch, about 3 grams of ground flax seed, about 6 grams of almond flour, about 3 grams of oat bran, about 58 grams of bananas, about 7 grams of fructose, about ½ teaspoon of vanilla, about ¼ teaspoon of allspice, about ¼ teaspoon of nutmeg, about ¼ teaspoon of baking powder, about ⅛ teaspoon of salt, and about 1/16 teaspoon of baking soda, so as to result in about one serving.

9. The method of claim 5, wherein said muffin is a blueberry muffin comprising about 50 grams of egg whites, about 16.5 grams of whey protein, about 17 grams of rolled oats, about 3.5 grams of cornstarch, about 3 grams of ground flax seed, about 6 grams of almond flour, about 5 grams of oat bran, about 20 grams of apple sauce, about 42 grams of blueberries, about 8.5 grams of fructose, about ¼ teaspoon of baking powder, about ⅛ teaspoon of salt, and about 0.25 grams of xanthan gum, so as to result in about one serving.

10. The method of claim 5, wherein said muffin is a cherry-vanilla muffin comprising about 50 grams of egg whites, about 16.5 grams of whey protein, about 16 grams of rolled oats, about 3 grams of cornstarch, about 3 grams of ground flax seed, about 6 grams of almond flour, about 5 grams of oat bran, about 15 grams of yogurt, about 58 grams of cherries, about 6 grams of fructose, about one teaspoon vanilla, about ¼ teaspoon baking powder, about ⅛ teaspoon salt, and about 1/16 teaspoon baking soda, so as to result in about one serving.

11. The method of claim 5, wherein said chicken potato soup comprises about 56 grams of chicken breast, cooked by dry heat without skin, about 142 grams of a boiled potato, about 85 grams of a raw carrot, about 56 grams of diced raw celery, about 40 grams of a chopped raw onion, about 2.5 grams of extra virgin olive oil, about 2 chicken bouillon cubes, and about 2¼ cups water, wherein the chicken breast, potato, carrot, celery, onion, olive oil, bouillon, and water are heated to result in about one single serving.

12. The method of claim 5, wherein said chicken potato and pasta soup comprises about 56 grams of chicken breast, about 71 grams of a boiled potato, about 42 grams of pasta-durum semolina, about 85 grams of a carrot, about 56 grams of celery, about 40 grams of an onion, about 2½ grams of olive oil, about 2 chicken bouillon cubes, about 2¼ cups of water, wherein the chicken breast, potato, pasta, carrot, celery, onion, olive oil, bouillon cubes, and water are heated to result in about one single serving.

13. The method of claim 5, wherein said turkey chili comprises about 2 ounces lean ground turkey, about 5.9 ounces of kidney beans, about 2.81 ounces of tomato sauce, about 3.62 ounces of diced tomatoes, about 2 ounces of an onion, and about 0.24 ounces of chili seasoning to result in about one single serving.

14. The method of claim 5, wherein turkey chili and pasta comprises about 2.5 ounces lean ground turkey, about 1.5 ounces of pasta, about 2.38 ounces of kidney beans, about 3.75 ounces of tomato sauce, about 3.63 ounces of diced tomatoes, about 0.75 ounces of green peppers, about 0.75 ounces of an onion, and about 0.24 ounces of chili seasoning to result in about one single serving.

15. The method of claim 5, wherein said chicken whole wheat burrito comprises about 43 grams of chicken breast roasted without skin, about 65 grams of a whole wheat tortilla, about 35 grams of cooked long grain rice, about 28 grams of cooked black beans, and about 99 grams of raw chopped tomatoes so as to result in about one single serving.

16. The method of claim 15, wherein said whole wheat burrito substitutes about 1.75 ounces of lean sirloin beef or about 2 ounces of Mahi Mahi fish for said chicken breast.

17. The method of claim 5, wherein said whole wheat pita pizza comprises about 1.5 ounces of cubed chicken breast, about 2.25 ounces of pita bread, about 2 ounces of low fat pasta sauce, about 2.25 ounces of sliced mushrooms, and about 0.5 ounce of reduced fat cheese so as to result in about one single serving.

18. The method of claim 17, wherein said chicken breast can be substituted with about 2 ounces of lean turkey sausage, about 2 ounces of lean ground turkey, or about 3 ounces of Canadian bacon.

19. The method of claim 17, wherein said mushrooms can be substituted with about 1.4 ounces of onion, about 1.8 ounces of sweet peppers, or about 2.4 ounces of tomato.

20. The method of claim 5, wherein said whole wheat pizza comprises about 11 grams of mushrooms, about 17 grams of sweet pepper, about 17 grams of onion, about 51 grams of low-fat pasta/pizza sauce, about 56 grams of whole wheat pizza crust, about 56 grams of lean turkey sausage, and about 0.80 grams of fat-free mozzarella cheese so as to result in about one single serving.

21. The method of claim 20, wherein said lean turkey sausage can be substituted with about 2 ounces of ground turkey or 1.5 ounces of chicken breast.

22. The method of claim 5, wherein said fruit yogurt comprises about 19 grams of whey protein, about 220 grams of low-fat vanilla yogurt, about 99 grams of strawberries, and about 4 grams of fructose so as to result in about one single serving.

23. The method of claim 22, wherein said strawberries can be substituted with about 50 grams of apples, about 54 grams of blueberries, about 45 grams of cherries, about 70 grams of peaches, about 54 grams of pineapple, and about 61 grams of raspberries.

24. A food stuff, comprising:
about 25-35% protein;
about 50-55% carbohydrate; and
about 15-20% fat.

25. The method of claim 24, wherein said food is selected from the group a muffin, fruit yogurt, chicken potato soup, chicken potato & pasta soup, turkey chili, turkey chili & pasta, chicken whole wheat burrito, whole wheat pizza, or whole wheat pita pizza.

26. The method of claim 25, wherein said muffin is a pumpkin muffin comprising about 50 grams of egg whites, about 16.5 grams of whey protein, about 16.5 grams of rolled oats, about 3 grams of cornstarch, about 3 grams ground flax seed, about 6 grams of almond flour, about 2.5 grams of oat bran, about 115 grams of pumpkin, about 7.5 grams of fructose, about 1.5 grams of honey, about ⅔ teaspoon of pumpkin pie spice, about ⅔ teaspoon of cinnamon, about ¼ teaspoon of baking powder, about ⅛ teaspoon salt, and about 1/16 teaspoon of baking soda, so as to result in about one serving.

27. The method of claim 25, wherein said muffin is an apple cinnamon muffin, comprising about 50 grams of egg whites, about 17 grams of whey protein, about 16 grams of rolled oats, about 3 grams of cornstarch, about 3 grams of ground flax seed, about 6 grams of almond flour, about 3 grams of oat bran, about 30 grams of apple sauce, about 56 grams of apples, about 8 grams of fructose, about ⅔ teaspoon of apple pie spice, about ⅔ teaspoon of cinnamon, about ¼ teaspoon of baking powder, about ⅛ teaspoon of salt, and about 1/16 teaspoon of baking soda, so as to result in about one serving.

28. The method of claim 25, wherein said muffin is a banana-nutmeg muffin comprising about 50 grams of egg whites, about 16.5 grams of whey protein, about 16.5 grams of rolled oats, about 2.5 grams of cornstarch, about 3 grams of ground flax seed, about 6 grams of almond flour, about 3 grams of oat bran, about 58 grams of bananas, about 7 grams of fructose, about ½ teaspoon of vanilla, about ¼ teaspoon of allspice, about ¼ teaspoon of nutmeg, about ¼ teaspoon of baking powder, about ⅛ teaspoon of salt, and about 1/16 teaspoon of baking soda, so as to result in about one serving.

29. The method of claim 25, wherein said muffin is a blueberry muffin comprising about 50 grams of egg whites, about 16.5 grams of whey protein, about 17 grams of rolled oats, about 3.5 grams of cornstarch, about 3 grams of ground flax seed, about 6 grams of almond flour, about 5 grams of oat bran, about 20 grams of apple sauce, about 42 grams of blueberries, about 8.5 grams of fructose, about ¼ teaspoon of baking powder, about ⅛ teaspoon of salt, and about 0.25 grams of xanthan gum, so as to result in about one serving.

30. The method of claim 25, wherein said muffin is a cherry-vanilla muffin comprising about 50 grams of egg whites, about 16.5 grams of whey protein, about 16 grams of rolled oats, about 3 grams of cornstarch, about 3 grams of ground flax seed, about 6 grams of almond flour, about 5 grams of oat bran, about 15 grams of yogurt, about 58 grams of cherries, about 6 grams of fructose, about one teaspoon vanilla, about ¼ teaspoon baking powder, about ⅛ teaspoon salt, and about 1/16 teaspoon baking soda, so as to result in about one serving.

31. The method of claim 25, wherein said chicken potato soup comprises about 56 grams of chicken breast, cooked by dry heat without skin, about 142 grams of a boiled potato, about 85 grams of a raw carrot, about 56 grams of diced raw celery, about 40 grams of a chopped raw onion, about 2.5 grams of extra virgin olive oil, about 2 chicken bouillon cubes, and about 2¼ cups water, wherein the chicken breast, potato, carrot, celery, onion, olive oil, bouillon, and water are heated to result in about one single serving.

32. The method of claim 25, wherein said chicken potato and pasta soup comprises about 56 grams of chicken breast, about 71 grams of a boiled potato, about 42 grams of pasta-durum semolina, about 85 grams of a carrot, about 56 grams of celery, about 40 grams of an onion, about 2½ grams of olive oil, about 2 chicken bouillon cubes, about 2¼ cups of water, wherein the chicken breast, potato, pasta, carrot, celery, onion, olive oil, bouillon cubes, and water are heated to result in about one single serving.

33. The method of claim 25, wherein said turkey chili comprises about 2 ounces lean ground turkey, about 5.9 ounces of kidney beans, about 2.81 ounces of tomato sauce, about 3.62 ounces of diced tomatoes, about 2 ounces of an onion, and about 0.24 ounces of chili seasoning to result in about one single serving.

34. The method of claim 25, wherein turkey chili and pasta comprises about 2.5 ounces lean ground turkey, about 1.5 ounces of pasta, about 2.38 ounces of kidney beans, about 3.75 ounces of tomato sauce, about 3.63 ounces of diced tomatoes, about 0.75 ounces of green peppers, about 0.75 ounces of an onion, and about 0.24 ounces of chili seasoning to result in about one single serving.

35. The method of claim 25, wherein said chicken whole wheat burrito comprises about 43 grams of chicken breast roasted without skin, about 65 grams of a whole wheat tortilla, about 35 grams of cooked long grain rice, about 28 grams of cooked black beans, and about 99 grams of raw chopped tomatoes so as to result in about one single serving.

36. The method of claim 35, wherein said whole wheat burrito substitutes about 1.75 ounces of lean sirloin beef or about 2 ounces of Mahi Mahi fish for said chicken breast.

37. The method of claim 25, wherein said whole wheat pita pizza comprises about 1.5 ounces of cubed chicken breast, about 2.25 ounces of pita bread, about 2 ounces of low fat pasta sauce, about 2.25 ounces of sliced mushrooms, and about 0.5 ounce of reduced fat cheese so as to result in about one single serving.

38. The method of claim 37, wherein said chicken breast can be substituted with about 2 ounces of lean turkey sausage, about 2 ounces of lean ground turkey, or about 3 ounces of Canadian bacon.

39. The method of claim 37, wherein said mushrooms can be substituted with about 1.4 ounces of onion, about 1.8 ounces of sweet peppers, or about 2.4 ounces of tomato.

40. The method of claim 25, wherein said whole wheat pizza comprises about 11 grams of mushrooms, about 17 grams of sweet pepper, about 17 grams of onion, about 51 grams of low-fat pasta/pizza sauce, about 56 grams of whole wheat pizza crust, about 56 grams of lean turkey sausage, and about 0.80 grams of fat-free mozzarella cheese so as to result in about one single serving.

41. The method of claim 40, wherein said lean turkey sausage can be substituted with about 2 ounces of ground turkey or 1.5 ounces of chicken breast.

42. The method of claim 25, wherein said fruit yogurt comprises about 19 grams of whey protein, about 220 grams of low-fat vanilla yogurt, about 99 grams of strawberries, and about 4 grams of fructose so as to result in about one single serving.

43. The method of claim 42, wherein said strawberries can be substituted with about 50 grams of apples, about 54 grams of blueberries, about 45 grams of cherries, about 70 grams of peaches, about 54 grams of pineapple, and about 61 grams of raspberries.
